(19) United States
(12) Patent Application Publication
(10) Pub. No.: US 2024/0366237 A1
(43) Pub. Date: Nov. 7, 2024

(54) Fixed Angle Tool Holder

(71) Applicant: Howmedica Osteonics Corp., Mahwah, NJ (US)

(72) Inventor: Peter Tulkis, Paramus, NJ (US)

(21) Appl. No.: 18/652,259

(22) Filed: May 1, 2024

Related U.S. Application Data
(60) Provisional application No. 63/464,311, filed on May 5, 2023.

(51) Int. Cl. A61B 17/16; A61B 17/00; A61B 17/88

(52) U.S. Cl. CPC A61B 17/1617; A61B 17/8875; A61B 2017/00738

(57) ABSTRACT

A tool assembly includes a variable angle tool that has first and second drive members with respective first and second axes. The second drive member is moveably connected to the first drive member so as to form a variable angle therebetween. The assembly also includes a fixed angle holder that has a first support member, a second support member, and a bridge. The first and second support members respectively rotatably hold the first and second drive members. The bridge is connected to and extends between the first and second support members such that the first and second drive members are held in a fixed position when the first and second drive members are held by the respective first and second drive members.

### CROSS REFERENCE TO RELATED APPLICATION

This application claims the benefit of the filing date of U.S. Provisional Patent Application No. 63/464,311, filed May 5, 2023, the disclosure of which is hereby incorporated by reference herein in its entirety.

### BACKGROUND OF THE INVENTION

Orthopedic surgery often involves the use of bone screws for various purposes, such as securing an implant to bone or securing multiple pieces of bone to each other, for example. Some bone screws may be self-tapping such they can be inserted into bone without the aid of any previously formed pilot hole. On the other hand, other screws may require that a pilot hole be drilled prior to insertion to help guide the screw into the bone along a desired trajectory and to help prevent the bone from cracking as the screw is introduced into the bone. In any event, drilling a hole and driving a screw typically requires rotation of a drill bit/screw about a drive axis and a minimum level of force applied along the drive axis to enable translation of the drill bit/screw along such axis. This can be complicated by limited access and awkward screw placement locations which are commonly presented in orthopedic surgery.

Fixed angled drills and drivers that are currently available for orthopedic surgery may be sufficient to address the problems mentioned above. However, at least one drawback associated with these instruments is that they often rely on complicated and expensive internal components, such as gears and ball bearings, that typically must be hermetically sealed to prevent exposure to the wound environment and be able to withstand the harsh conditions of cleaning and steam sterilization. Additionally, multiple drills and drivers with different angles may be needed in an operating theater to address different scenarios which may arise and that may not be able to be addressed with any one fixed angled driver of a particular angle, which may further increase costs.

Drill/driver guides used in conjunction with variable angle drills and drivers may help overcome some of the issues mentioned above with respect to fixed angled drills and drivers. However, such guides typically only guide the tool bit at the very end of the tool. As such, the variable angle joint can be unstable since the surgeon's other hand is occupied holding the guide thereby potentially leading to a less than optimal drill trajectory, cam-out, screw head stripping, and the like. Additionally, it may be difficult to provide consistent driving force along a drive axis as it may be difficult to maintain a consistent drill/drive angle throughout the operation. Therefore, further improvements are desirable.

### BRIEF SUMMARY OF THE INVENTION

In one aspect of the present disclosure, a tool assembly includes a variable angle tool that has a first drive member that defines a first drive axis and a second drive member that defines a second drive axis. The second drive member is connected to the first drive member such that the second drive member is moveable relative to the first drive member from a first position in which the first and second drive axes form a first angle and a second position in which the first and second drive axes form a second angle different than the first angle. The assembly also includes a fixed angle holder that has a first support member, a second support member, and a bridge. The first support member and the second support member are respectively configured to rotatably hold the first and second drive members. The bridge is connected to and extends between the first and second support members such that the first and second drive members are held in the first position when the first and second drive members are rotatably held by the respective first and second drive members.

Additionally, the first and second drive members may be connected together such that rotating the first drive member about the first drive axis rotates the second drive member about the second drive axis. The first drive member may include a first connection end, the second drive member may include a second connection end, and the first and second connection ends may form a joint with at least two degrees of freedom. The joint may include a spherical joint, cylindrical joint, and/or a universal joint. Also, the joint may be positioned between the first and second support members so that the bridge spans along one side of the joint.

Also, the first drive member may include a first end and a second end. The first end may be connectable to a torque applying device, the second end may be connected to the second drive member, and the first support member may define a longitudinal opening that extends between first and second ends of the first support member. The longitudinal opening may be configured to rotatably receive the first drive member such that the first end of the first drive member extends from the first end of the first support member, and the second end of the first drive member may extend from the second end of the first support member.

Continuing with this aspect, the first support member may define a longitudinal slot that extends through an exterior thereof and may be in communication with the longitudinal opening. The longitudinal slot may be configured to receive the first drive member so that the first drive member can be deposited into the longitudinal opening from the longitudinal slot in a direction perpendicular to the first support member. The first support member may include a retaining mechanism moveable from a first position to a second position to secure the first drive member within the longitudinal opening. The retaining mechanism may be a latch moveably disposed within the first end of the first support member. The latch may include a protrusion and may be connected to the first support member via a spring which biases the latch in the first position. The protrusion may overlap the longitudinal opening thereby prohibiting the first drive member from being removed therefrom when disposed therein. The protrusion may also include a ramp surface configured to deflect the latch to the second position when the first drive member is passed through the longitudinal slot and into the longitudinal opening.

Further, the second drive member may include a first end and a second end. The first end may be connectable to the second end of the first drive member, and the second end of the second drive member may include at least one of a drill bit or screwdriver bit. The first drive member may define a longitudinal opening that extends through it. The longitudinal opening may be configured to rotatably receive at least one of the drill bit or screw driver bit.

In another aspect of the present disclosure, a tool assembly includes a variable angle tool that has a first drive member and a second drive member. The first drive member has a first end and a second end and defines a first drive axis. The second drive member has a first end and a second end and defines a second drive axis. The second end of the first drive member is connected to the first end of the second drive member to form a joint that has at least two degrees of freedom. The assembly also includes a fixed angle holder that has a handle, a bushing, and a bridge. The handle has a first longitudinal opening that is configured to rotatably receive the first drive member. The bushing has a second longitudinal opening that is configured to rotatably receive the second drive member. The bridge is connected to and extends between the handle and bushing.

Additionally, the second end of the second drive member may include a drill bit or screwdriver bit, and the second longitudinal opening of the bushing may be configured to rotatably receive the drill bit or screwdriver bit. The first longitudinal opening of the handle may define a first longitudinal axis, the second longitudinal opening of the bushing may define a second longitudinal axis, and the bridge may connect to the handle and the bushing so that the first and second longitudinal axes intersect at a fixed angle. The fixed angle may be 45 degrees. Also, the joint may be positioned between the handle and the bushing when the first and second drive members are respectively connected thereto.

### BRIEF DESCRIPTION OF THE DRAWINGS

The features, aspects, and advantages of the present invention will become better understood with regard to the following description, appended claims, and accompanying drawings in which:

FIG. 1A is perspective view of a variable angle tool according to an embodiment of the present disclosure.

FIG. 1B is a close up view of a distal end of the variable angle tool of FIG. 1A with a different end effector.

FIG. 1C is an exploded view of a distal end of the variable angle tool of FIG. 1A.

FIG. 1D is a side view of the variable angle tool of FIG. 1A.

FIG. 1E is a cross-sectional view of the distal end of the variable angle tool taken along a line E-E of FIG. 1C.

FIG. 2A is a perspective view of a fixed angle holder according to an embodiment of the present disclosure.

FIG. 2B is a rear perspective view of the fixed angle holder of FIG. 2A.

FIG. 2C is a rear view of the fixed angle holder of FIG. 2A.

FIGS. 3A-3C illustrate a method of assembling the variable angle tool of FIG. 1A with the fixed angle holder of FIG. 2A.

FIG. 3D is a cross-sectional view of a tool assembly including the variable angle tool of FIG. 1A with the fixed angle holder of FIG. 2A taken along a midline of the tool assembly.

FIG. 3E is an enhanced view of a distal end of the tool assembly of FIG. 3D.

### DETAILED DESCRIPTION

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended. As used herein, the terms “substantially,” “generally,” “approximately,” and “about” are intended to mean that slight deviations from absolute are included within the scope of the term so modified, such as deviations of up to 10% greater or lesser than absolute.

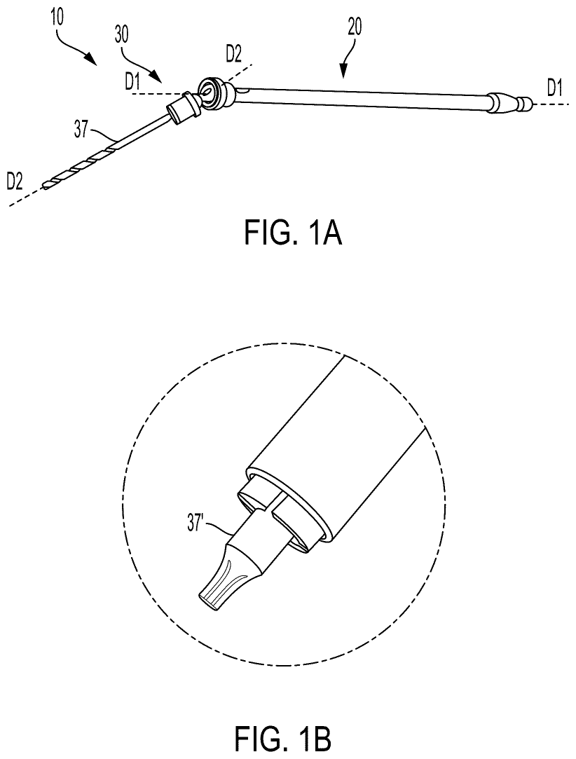

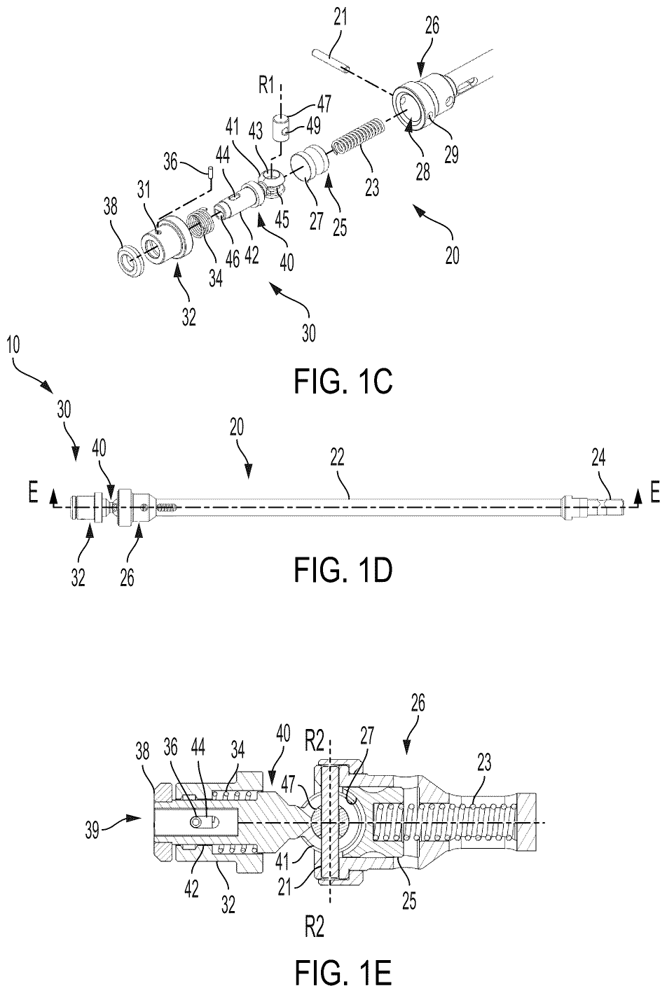

FIGS. 1A-1E depict a variable angle tool 10 according to an embodiment of the present disclosure. Variable angle tool 10 generally includes a first drive member 20 and a second drive member 30. As discussed in more detail below, first and second drive members 20, 30 are connected together to form a joint that allows second drive member 30 to be rotated relative to first drive member 20 so that an end of second drive member 20, which may include a drill bit or screwdriver bit, can be oriented in a position that makes it easier for a surgeon to access hard to reach places.

First drive member or proximal drive member 20 includes an elongate shaft 22 that defines a first drive axis D1. A first end or proximal end 24 of first drive member 20 is configured to connect to a torque applying device, such as a power drill, for example. A connection end 26 is disposed at a second end or distal end of shaft 22 and is configured to connect to second drive member 30. In the particular embodiment depicted, connection end 26 is generally cylindrical and includes a chamber 28. Connection end 26 also includes a pin 21, spring 23, and plunger 25, as best shown in FIGS. 1C and 1E. Plunger 25 has a spherical recess 27 and is disposed within chamber so that spherical recess 27 faces distally. Spring 23 is also disposed within chamber 28 and biases plunger 25 in a distal direction. Connection end 26 has a pin hole 29 extending transverse to first drive axis D1 and connects first drive member 20 to second drive member 30, as described below.

Second drive member or distal drive member 30 generally includes a first end and a second end and defines a second drive axis D2 extending from the first end to the second end of second drive member 30. The first end is a connection end that is configured to rotatably connect to first drive member 20. In the particular embodiment depicted, first end includes a connector 40 and a bearing 47. Connector 40 includes a ball 41 and a post 42 extending from ball 41. Post 42 has a guide slot 44 extending through a side thereof, and a bit opening 46 extending through a distal end thereof which is generally semi-circular. Ball 41 is spherically shaped and is configured to be received within spherical recess 27 of plunger 25 and to articulate therein as a spherical joint. Ball 41 includes a bore 43 that defines a first rotational axis R1 that extends transverse to second drive axis D2. A circumferential slot 45 extends through ball 41 and partially about axis R1, as shown in FIG. 1B. Bearing 47 is cylindrical and is configured to be rotatably received within bore 43 to form a cylindrical joint with a rotational degree of freedom about R1. A pin 49 opening extends through bearing 47 so that when bearing 47 is received within bore 43, pin opening 49 aligns with circumferential slot 45.

The second or distal end of second drive member 30 is an effector end and, as such, effectuates a desired operative function, such as drilling and/or driving a screw. As such, the second end of second drive member includes an effector 37, such as the drill bit 37 of FIG. 1A or the screwdriver bit 37′ of FIG. 1B, for example. The second end of second drive member 30 also includes components that allow such effector 37 to be modularly connected to variable angle tool 10. In the particular embodiment depicted, such components generally include a pull sleeve 32, a spring 34, and an end plate 38. As shown in FIG. 1C, pull sleeve 32 and spring 34 are positioned over post 42 of connector 40 and connected thereto via pin 36 which is received within slot 44 of post 42. This allows pull sleeve 32 to slide proximally-distally along post 42 to capture and release effector 37. End plate 38 is press-fit to post 42 to further secure pull sleeve 32 to connector 40.

As shown in FIG. 1E, first drive member 20 and second drive member 30 connect together to form an articulating joint. In particular, ball 41 is received within socket 27 of plunger 25. Additionally, bearing 47 is received within bore 43 of ball 41 and is secured thereto via pin 21 which extends through bearing 47 and circumferential slot 45 and is secured to pin hole 29 of connection end 26. In this configuration, joint is rotatable about a first rotation axis R1, which is defined by bearing 47, and second rotation axis R2, which is defined by pin 21, as shown in FIGS. 1C and 1E. Thus, the joint has two degrees of rotational freedom and is defined by a spherical joint and two cylindrical joints that constrain the spherical joint. This joint allows rotation applied to first drive member 20 about first drive axis D1 to be transferred to second drive member 30 about second drive axis D2 while maintaining a desired angle between them provided that first and second drive members 20, 30 are held at the desired angle.

The joint formed between first and second drive members 20, 30 is exemplary of a variable angle joint. Therefore, it should be understood that other variable angle joint configurations that have at least two degrees of freedom may be utilized. Such joints may include spherical joints, universal joints, cylindrical joints, and the like.

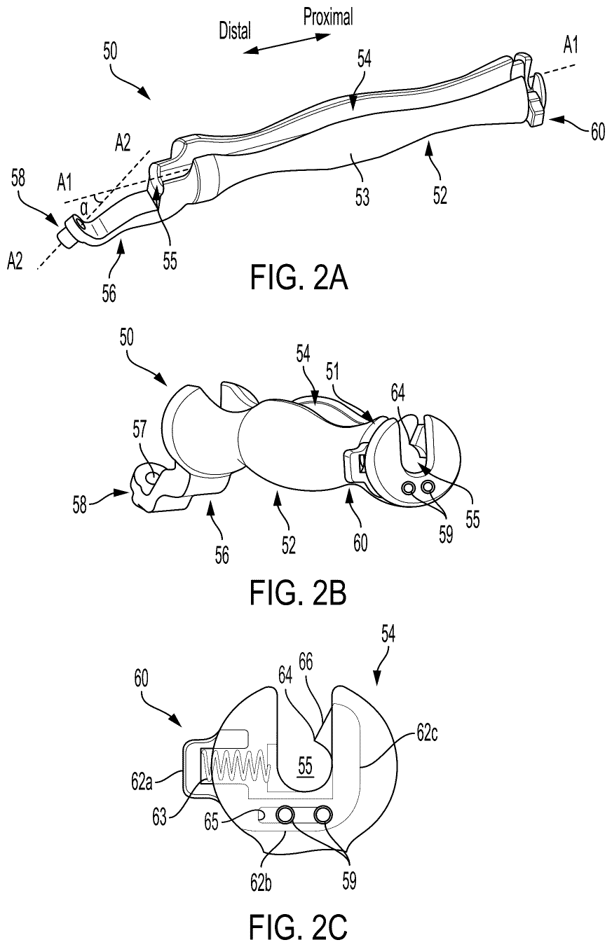

FIGS. 2A-2C depict a fixed angle holder 50 according to an embodiment of the disclosure. Fixed angle holder 50 is configured to modularly connect to variable angle tool 10 and to hold first and second drive members 20, 30 at a desired fixed angle. Fixed angle holder 50 generally includes a first support member 52, a second support member 58, and a bridge 56 connected to and extending between the first and second support members 52, 58.

First support member 52 is a handle that has an exterior surface 53 ergonomically shaped to be grasped by a hand. A first longitudinal opening 55 configured to receive first drive member 20 extends through handle 52 and defines a first longitudinal axis A1. A longitudinal slot 54 extends from exterior surface 53 of handle 52 and along its length. Longitudinal slot 54 communicates with longitudinal opening 55, as shown in FIG. 2A. A transverse slot 51 extends into handle 52 at a first or proximal end thereof and extends in a direction transverse to first longitudinal axis A1.

A latch or shuttle 60 is disposed within transverse slot 51 and is moveable from a first position to a second position. As best shown in FIG. 2C, latch 60 includes a first member 62a, second member 62b, and third member 62c. First member 62a is connected to second member 62b, and second member 62b is connected to third member 62c so that all of members 62a-c together form a general U-shape. A spring 63 is connected to handle 52 and to first member 62a. Such spring 63 biases latch 60 into the first position. Second member 62b includes a guide channel 65 and is connected to handle 52 via guide pins 59 extending through guide channel 65. As latch 60 is moved between first and second positions, guide pins 59 slide within channel 65. Third member 62c includes a protrusion 64 that extends radially inwardly and over longitudinal opening 55 when latch 60 is in the first position, as shown in FIG. 2C. Protrusion 64 has a deflection surface 66 facing longitudinal slot 54. Deflection surface may be a ramped surface, for example. As such, when first drive member 20 is loaded through slot 54, it contacts deflection surface 66 and deflects latch 60 to the second position when first drive member 20 is loaded into longitudinal opening 55 via longitudinal slot 54.

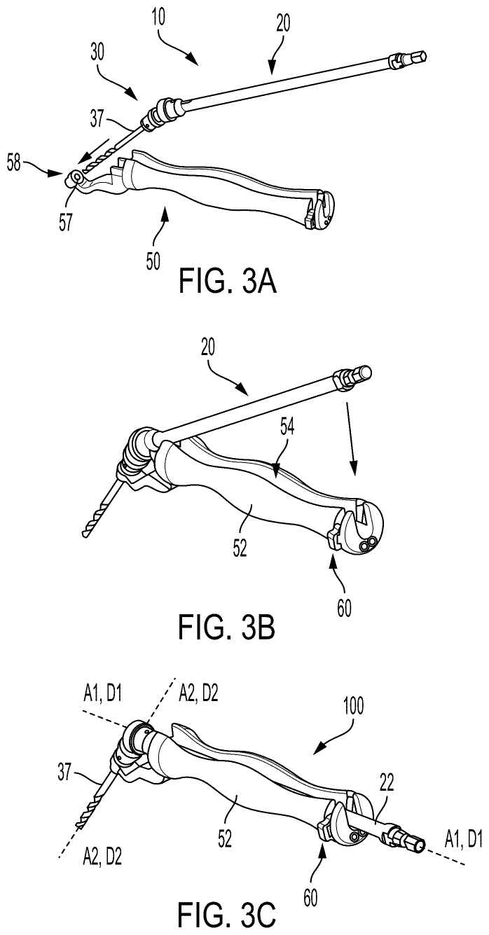

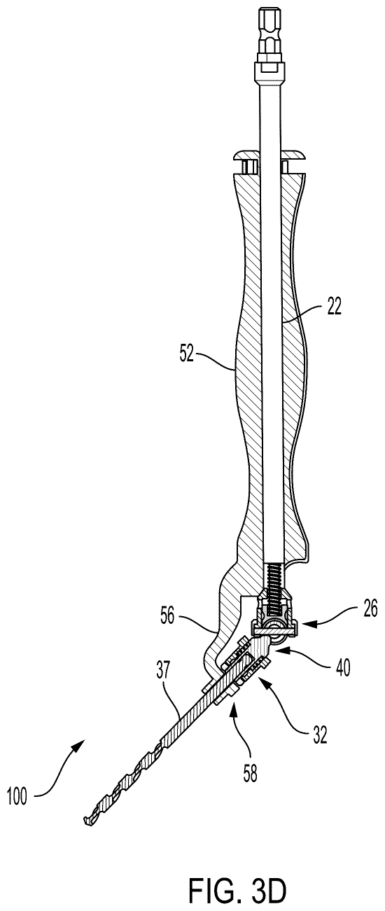

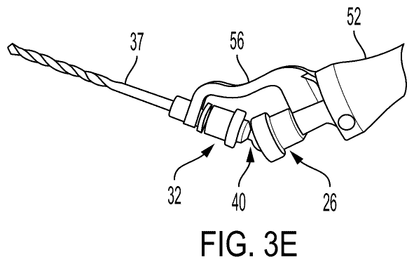

Second support member 58 is a bushing that includes a longitudinal opening 57 extending therethrough which defines a second longitudinal axis A2. As shown in FIG. 3E, bushing 58 is configured to rotatably receive effector 37. Opening 57 may also be configured to rotatably receive pull sleeve 32. However, keeping pull sleeve 32 out of bushing 58 allows pull sleeve 32 to be activated while variable angle drill 10 is connected to fixed angle holder 50 in order to swap out effectors 37, as needed.

Bridge or arm 56 is connected to the distal end of first support member 52 and a proximal end of second support member 58. Bridge 56 is canted so that first longitudinal axis A1 of first support member 52 is oriented at a fixed angle α relative to second longitudinal axis A2 of second support member 30, as shown in FIG. 2A.

FIGS. 3A-3C depict a method of assembling a tool assembly 100 that includes variable angle tool 10 and fixed angle holder 50. Variable angle tool 10 may be modularly connected to fixed angle holder 50 such that the method of assembling tool assembly 100 may be performed in an operating theater. As shown in FIGS. 3A and 3B, assembly of tool assembly 100 begins with inserting the second end of second drive member 30 through bushing 58 so that effector 37 extends therefrom. As shown in FIGS. 3B and 3C, first drive member 20 is then loaded into first support member 52 by pushing first drive member 20 through longitudinal slot 54 and into longitudinal opening 55. As first drive member 20 is being pushed through longitudinal slot 54, shaft 22 of driver contacts deflection surface 66 of latch 60 which deflects latch 60 to its second position thereby allowing passage of first drive member 50 into longitudinal opening 55. Once first drive member 20 passes protrusion 64, the bias of spring 63 pushes latch 60 back to the first position. In this position, protrusion 64 prevents first drive member 20 from being removed from first support member 52 without deliberate intervention in the form of pushing latch, which extends laterally from handle 52, against the bias of spring 63 and back to the second position. Although, first drive member 20 is constrained by latch 60 in this manner, first drive member 20 may be freely rotated about first drive axis D1.

As shown in FIGS. 3C-3E, when tool assembly 100 is assembled, first and second drive members 20, 30 respectively align with first and second support members 52, 58 such that first drive axis D1 is coaxial with first longitudinal axis A1 and second drive axis D2 is coaxial with second longitudinal axis A2, as shown in FIG. 3C. Proximal end 24 of first drive member 20 extends from the proximal end of first support member 52 so that a torque applying device can be connected thereto. The joint of tool 10 is positioned between first and second support members 52, 58 while bridge 56 spans the joint at one side thereof, as best shown in FIG. 3D. This configuration helps stabilize the joint particularly as a surgeon pushes effector 37 along second drive axis D2 to help effectuate drilling/driving. Fixed tool holder 50 secures variable angle tool 10 at fixed angle α at least by virtue of ridged bridge 56 extending between first and second support members 52, 58.

As mentioned above, since variable angle tool 10 may be modularly connected to fixed angle holder 50 in the operating theater as needed, a kit may be provided to afford a variety of options for a surgeon. In this regard, a kit may include variable angle tool 10 and a plurality of fixed angle holders 50 each with a different fixed angle. For example, a kit may include a plurality of fixed angled holders 50 such that a first fixed angle holder has a fixed angle of 30 degrees, a second fixed angle holder with a fixed angle of 45 degrees, and a fixed tool holder with a fixed angle of 60 degrees. In other embodiments, fixed angle holders 50 may be provided such that the differences between the fixed angles of such holders are in 5-degree increments beginning with 30 degrees and ending with 60 degrees, for example.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A tool assembly, comprising:
a variable angle tool having a first drive member defining a first drive axis and a second drive member defining a second drive axis, the second drive member being connected to the first drive member such that the second drive member is moveable relative to the first drive member from a first position in which the first and second drive axes form a first angle and a second position in which the first and second drive axes form a second angle different than the first angle; and
a fixed angle holder having a first support member, a second support member, and a bridge, the first support member and the second support member being respectively configured to rotatably hold the first and second drive members, and the bridge being connected to and extending between the first and second support members such that the first and second drive members are held in the first position when the first and second drive members are rotatably held by the respective first and second support members.

2. The assembly of claim 1, wherein the first and second drive members are connected together such that rotating the first drive member about the first drive axis rotates the second drive member about the second drive axis.

3. The assembly of claim 1, wherein the first drive member includes a first connection end, the second drive member includes a second connection end, and the first and second connection ends form a joint having at least two degrees of freedom.

4. The assembly of claim 3, wherein the joint includes a spherical joint or a cylindrical joint.

5. The assembly of claim 3, wherein the joint includes a universal joint.

6. The assembly of claim 3, wherein the joint is positioned between the first and second support members and the bridge spans the along one side thereof.

7. The assembly of claim 1, wherein:
the first drive member includes a first end and a second end, the first end being connectable to a torque applying device, the second end being connected to the second drive member, and
the first support member defines a longitudinal opening extending between first and second ends of the first support member, the longitudinal opening being configured to rotatably receive the first drive member such that the first end of the first drive member extends from the first end of the first support member, and the second end of the first drive member extends from the second end of the first support member.

8. The assembly of claim 7, wherein the first support member defines a longitudinal slot extending through an exterior thereof and being in communication with the longitudinal opening, the longitudinal slot being configured to receive first drive member so that the first drive member can be deposited into the longitudinal opening from the longitudinal slot in a direction perpendicular to the first support member.

9. The assembly of claim 8, wherein the first support member includes a retaining mechanism moveable from a first position to a second position to secure the first drive member within the longitudinal opening.

10. The assembly of claim 9, wherein the retaining mechanism is a latch moveably disposed within the first end of the first support member.

11. The assembly of claim 10, wherein the latch includes a protrusion and is connected to the first support member via a spring which biases the latch in the first position wherein the protrusion overlaps the longitudinal opening prohibiting the first drive member from being removed therefrom when disposed therein.

12. The assembly of claim 11, wherein the protrusion has a deflection surface configured to deflect the latch to the second position when the first drive member is passed through the longitudinal slot and into the longitudinal opening.

13. The assembly of claim 12, wherein the deflection surface is a ramped surface.

14. The assembly of claim 7, wherein the second drive member includes a first end and a second end, the first end being connected to the second end of the first drive member, and the second end of the second drive member including at least one of a drill bit or screwdriver bit.

15. The assembly of claim 14, wherein the first drive member defines a longitudinal opening extending therethrough, the longitudinal opening being configured to rotatably receive at least one of the drill bit or screw driver bit.

16. A tool assembly, comprising:
a variable angle tool having a first drive member and a second drive member, the first drive member having a first end and a second end and defining a first drive axis, the second drive member having a first end and a second end and defining a second drive axis, the second end of the first drive member connected to the first end of the second drive member to form a joint having at least two degrees of freedom; and
a fixed angle holder having a handle, a bushing, and a bridge, the handle having a first longitudinal opening configured to rotatably receive the first drive member, the bushing having a second longitudinal opening configured to rotatably receive the second drive member, and the bridge connected to and extending between the handle and bushing.

17. The assembly of claim 16, wherein the second end of the second drive member includes a drill bit or screwdriver bit, and the second longitudinal opening of the bushing is configured to rotatably receive the drill bit or screwdriver bit.

18. The assembly of claim 16, wherein the first longitudinal opening of the handle defines a first longitudinal axis, the second longitudinal opening of the bushing defines a second longitudinal axis, and the bridge connects to the handle and the bushing so that the first and second longitudinal axes intersect at a fixed angle.

19. The assembly of claim 18, wherein the fixed angle is 45 degrees.

20. The assembly of claim 16, wherein the joint is positioned between the handle and the bushing when the first and second drive members are respectively connected thereto.
